(19) United States
(12) Patent Application Publication
(10) Pub. No.: US 2017/0319230 A1
(43) Pub. Date: Nov. 9, 2017

(54) Skin surgery knife

(71) Applicant: Tetsunori KUNIMUNE, Burbank, CA (US)

(72) Inventor: Tetsunori KUNIMUNE, Burbank, CA (US)

(21) Appl. No.: 15/149,828

(22) Filed: May 9, 2016

(51) Int. Cl. A61B 17/3209; A61B 17/3211; A61B 17/00

(52) U.S. Cl. CPC A61B 17/32093; A61B 17/3211; A61B 2017/00424

(57) ABSTRACT

A skin surgery knife is provided. The skin surgery knife according to current invention has a handle and a blade. The blade has a point, sides, and an edge ground on both of the sides to form a cutting edge. The cutting edge has a section near the handle and another section near the point of the blade. The cutting edge has two different acute cutting angels. The cutting edge angle of the section near the handle is smaller than the cutting edge angle of section near the point of the blade.

### FIELD OF THE INVENTION

Current invention relates to a surgery knife comprising blade and handle, especially to a skin surgery knife which has more acute cutting angle at the section near the handle than the cutting edge angle at the section near the point of the blade.

### BACKGROUND OF THE INVENTION

Surgery knife has many different function based on the surgery. Some surgery is cut the bone and some surgery is taken out the organs etc. But, whatever the surgery is the surgery begins with cutting the skin at the most outer side. Until now most efforts were focused on how well the knife cut the skin smoothly without leaving scratch mark, which will leave grubby scar after sealing the surgery portion. In the surgical arts, where the material being cut is living tissue, a sharp smooth cutting edge in is of paramount interest. The work required to pass a knife through tissue results from many factors, including edge sharpness, force applied to the blade, drag force acting on the sides of the blade, and the like. The trauma caused to tissue from an incision results in increased time required for healing, increased chance for infection, a limitation to the size of physiological structures that can be incised accurately, and unsightly scarring. To make keener knife cutting edges were ground more sharply and evenly. Some of the cutting edges were metal coated by electro chemical method and some cutting edges were chemically treated and some are using abrasion method. Some invention provides a cutting instrument, wherein a maximum deviation from a line defined by two points on the ultimate edge of the cutting edge separated by 750 μm of any point on the ultimate edge of the cutting edge between the two points is about 1 μm or less. However, it was found that those sharp cutting edged knife cut leaves a mark not easily close. Contrarily the knife cut by the coarser blade close faster than the cut by the sharp cutting edge. To facilitate the closing of the surgery mark, a skin surgery knife with blunt cutting edge angle of section near the point of the blade was provided. The cutting edge angle of section near the handle is sharper and surface is even.

### DESCRIPTION OF THE PRIOR ARTS

United States Patent Application 20120124844 by Droese; Joachim disclose a chef's knife comprises a handle and a blade. The blade has a point sides, and an edge ground on both of the sides to form a cutting edge. The cutting edge has a section near the handle and a section near the point of the blade. The cutting edge has an acute cutting angle a that becomes continuously smaller from the section near the handle towards the section near the point of the blade. The cutting edge has a section near the handle and a section near the point of the blade and an acute cutting angle that is between 15 and 35 degrees in the section near the handle, is between 10 and 20 degrees in the section near the point of the blade, and defines a largest cutting angle and a smallest cutting angle, and a difference between the largest and smallest cutting angles is at least 8 degrees.

U.S. Pat. No. 1,820,234 to Nora Elsa LEES disclose a cutlery. The cutter and the like provide an unproved form of knife blade which will have a sharp cutting edge at the part most required or used for cutting without materially weakening the blade. The knife blade is hollow ground on one side along the cutting edge, the cross section area of the blade at or near the part mainly used for cutting process being substantially less than the cross sectional area of the manner. The reduction in the cross sectional area may be consequential to a tapering formation of knife blade where the blade tapers in the thickness longitudally as well as transversely or it may be achieved by grinding the hollow deeper at the requisite part.

U.S. Pat. No. 7,648,516 to Matsutani, et al. illustrated a medical knife has a cutting edge formed along the outer edge of a blade portion, and the thickness of the lower portion located under a boundary acting as a surface including the cutting edge is made thinner than that of an upper portion located on the boundary, or a lower surface is composed of the surface including the cutting edge. The surface of the upper portion on the above surface including the cutting edge is composed of a first slanting surface constituting the cutting edge, a second reflecting surface, and a flat surface composed of a non-reflecting surface as well as the lower portion has a lower slanting surface 10 and a lower flat surface 11 each composed of a reflecting surface. With the above arrangement, the medical knife can reduce the amount of reflected light when an operation such as an ophthalmic operation is executed under a microscope.

U.S. Pat. No. 5,713,915 Van Heugten, et al. illustrates a surgical knife blade of the type primarily intended for making incisions in the eye, though suitable for incising virtually any spherical surface, is disclosed. The blade is uniquely characterized by its construction to include cutting edge bevels of different transverse dimension on the anterior surface of the blade with respect to the transverse dimension of corresponding bevels on the posterior surface of the blade, such that the blade of this invention will incise a substantially straight, perpendicular cut into the eye or other spherical surface.

U.S. Pat. No. 7,037,175 to Abstract Spiro, et al. teaches a method for polishing a edge on a cutting instrument, comprising contacting a cutting edge of a cutting instrument with a polishing pad and a chemical-mechanical polishing composition comprising particles of an abrasive, and a liquid carrier, wherein the abrasive is suspended in the liquid carrier, and abrading at least a portion of the cutting edge to polish the cutting edge. That invention is directed to a method for polishing a cutting edge on a cutting instrument, comprising (i) providing a cutting instrument having a cutting edge, (ii) contacting the cutting edge with a polishing pad and a chemical-mechanical polishing composition comprising (a) particles it an abrasive, and (b) a liquid carrier, wherein the abrasive is suspended in the liquid carrier, (iii) moving the polishing pad relative to the cutting edge with the chemical-mechanical polishing composition therebetween, and (iv) abrading at least a portion of the cutting edge to polish the cutting edge. That invention further provides a cutting instrument, comprising a cutting instrument body having two flat faces and a direction of elongation and defining an ultimate edge, and having at least one cutting edge extending parallel to the direction of elongation, wherein a maximum deviation from a line defined by two points on the ultimate edge of the cutting edge separated by 5000 μm of any point on the ultimate edge of the cutting edge between the two points is about 4 μm or less.

However, none of the prior art disclose a skin surgery knife with blunt cutting edge angle of section near the point of the blade.

### SUMMARY

Surgery knife has many different function based on the surgery. Some surgery is cut the bone and some surgery is taken out the organs etc. But, whatever the surgery is the surgery begins with cutting the skin at most outer side. Until now most efforts were focused on how well the knife cut the skin smoothly without leaving scratch mark, which will leave grubby scar after sealing the surgery portion. In the surgical arts, where the material being cut is living tissue, a sharp and smooth cutting edge in is of paramount interest. The work required to pass a knife through tissue results from many factors, including edge sharpness, force applied to the blade, drug force acting on the sides of the blade, and the like. The trauma caused to tissue from an incision results in increased time required for healing, increased chance for infection, a limitation to the size of physiological structures that can be incised accurately, and unsightly scarring. To make keener knife cutting edges were ground more sharply and evenly. Some of the cutting edges were metal coated by electro chemical method and some cutting edges were chemically treated and some are using abrasion method. Some invention provides a cutting instrument, wherein a maximum deviation from a line defined by two points on the ultimate edge of the cutting edge separated by 750 μm of any point on the ultimate edge of the cutting edge between the two points is about 1 μm or less. However, it was found that those sharp cutting edged knife cut leaves a mark not easily close. Contrarily the knife cut by the coarser blade close faster than the cut by the sharp cutting edge. To facilitate the closing of the surgery mark, a skin surgery knife with blunt cutting edge angle of section near the point of the blade was provided. The cutting edge angle of section near the handle is sharper and surface is even. A skin surgery knife is provided. The skin surgery knife according to current invention has a handle and a blade. The blade has a point, sides, and an edge ground on both of the sides to form a cutting edge. The cutting edge has a section near the handle and another section near the point of the blade. The cutting edge has two different acute cutting angles. The cutting edge angle of the section near the handle is smaller than the cutting edge angle of section near the point of the blade.

### BRIEF DESCRIPTION OF THE DRAWINGS

FIG. 1 is the schematic sketch of the incision mark left with micrometer level sharpened scalpel after seven days.

FIG. 2 is the schematic sketch of the incision mark left with standard scalpel after four days.

FIG. 3 is the schematic sketch of the incision mark left with a skin surgery knife, modified scalpel, after five days.

FIG. 4 is a side view of a skin surgery knife according to the invention.

FIG. 5 is a cross-sectional view of the skin surgery knife of FIG. 4 along section line A-A′.

FIG. 6 is a cross-sectional view of the skin surgery knife of FIG. 4 along section line B-B′.

FIG. 7 is an enlarged, cross-sectional view of cutting edge portion of the skin surgery knife of FIG. 5.

FIG. 8 is an enlarged, cross-sectional view of cutting edge portion of the skin surgery knife of FIG. 6.

### DETAILED DESCRIPTION OF THE PREFERRED INVENTION

Skin surgery has many things to consider. Clean and quick closing of incision mark is both important. Among them clean closing of the skin is easy to control because tearing and snagging in the incision is mostly due to the coarse blade of the scalpel. That is why most of surgery knife, scalpel, was sharply bladed. Micrometer level sharpened scalpel left clean almost invisible. Trauma was considered as unnecessary pain, unnecessary medication and hospital stay. In addition to that excess trauma leads to wound swelling from blood engorgement. If the wound remains open, there is great risk of infection. Most of micrometer level sharpened scalpel is believed to have faster closing time because the macrophage density is lower than standard scalpel at 1^{st} date of surgery. But, that is because standard scalpel blade tears more skin than the micrometer level sharpened scalpel. This result does not show the micrometer level sharpened scalpel shows faster closing of the incision mark.

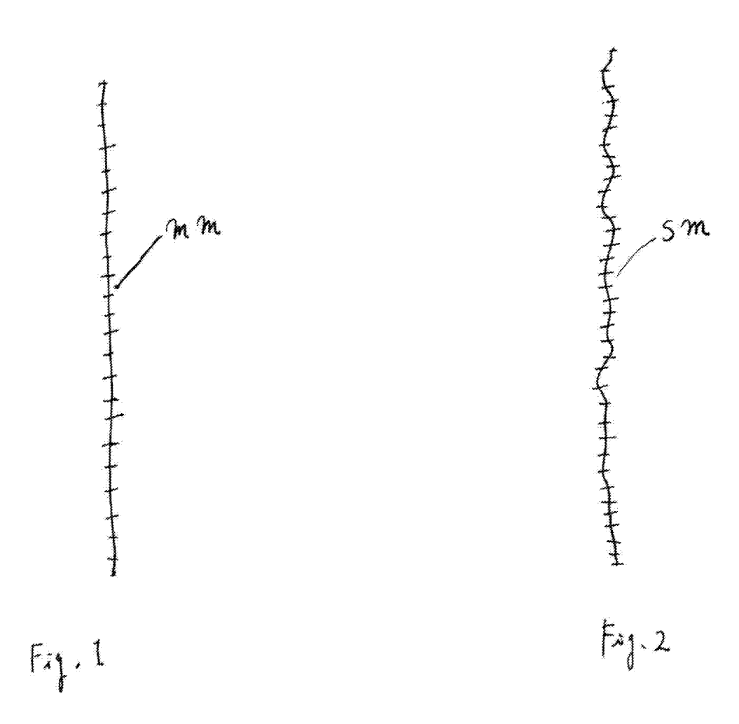

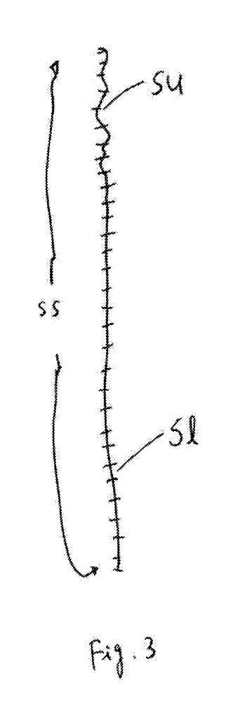

FIG. 1 is the schematic sketch of the incision mark (mm) left with micrometer level sharpened scalpel after seven days. Before seven days the incision mark (mm) was not closed and can be separated with had force. After seven days the incision mark (mm) was solidly closed. FIG. 2 is the schematic sketch of the incision mark (sm) left with standard scalpel after four days. Incision with standard scalpel left incision mark (sm) closed after four days.

This shows clearly that standard scalpel leaves rough and long incision line that increases the contact line, and facilitates the cure faster than clean surface of micrometer level sharpened scalpel. The high density of macrophage supports this activity.

FIG. 3 is the schematic sketch of the incision mark (ss) left with a skin surgery knife, modified scalpel, after five days. The upper part of the incision mark (su) is made by the blunt cutting edge near the point of the blade and lower part of the incision mark (sl) is made by the sharp cutting edge near the handle. Due to more active recovering activity along the standard scalpel cutting edge line, the upper part incision mark (su) close first, after four days, and facilitates closing of lower part of the incision mark (sl) in five days.

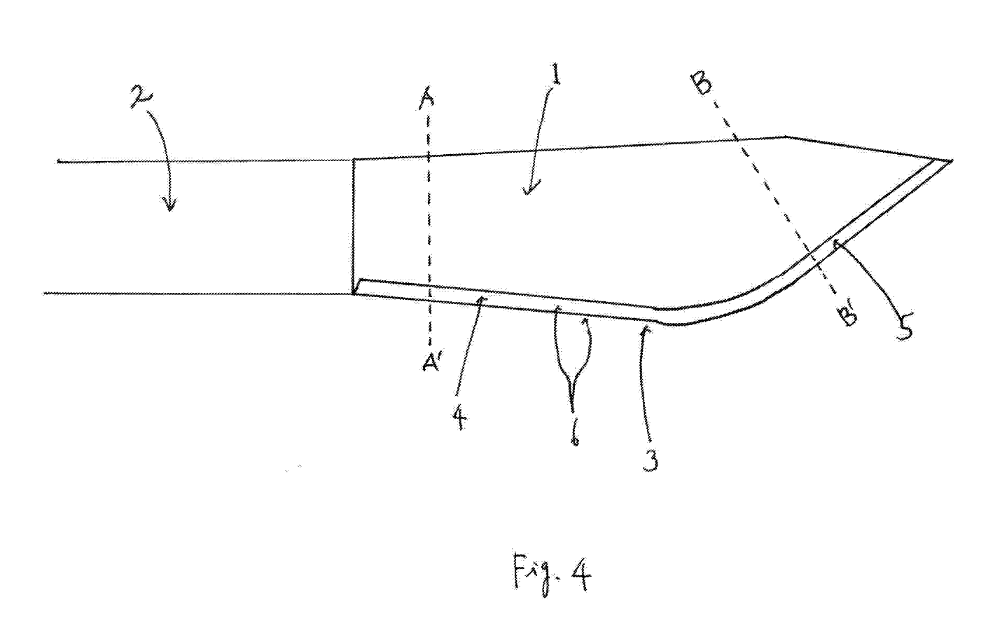

FIG. 4 is a side view of a skin surgery knife according to the invention. The skin surgery knife includes a blade (1) and a handle (2). The blade (1) has an edge (3) that is ground on both sides to form a cutting edges (4) and (5) with a facet (6) is formed on either side of the blade (1).

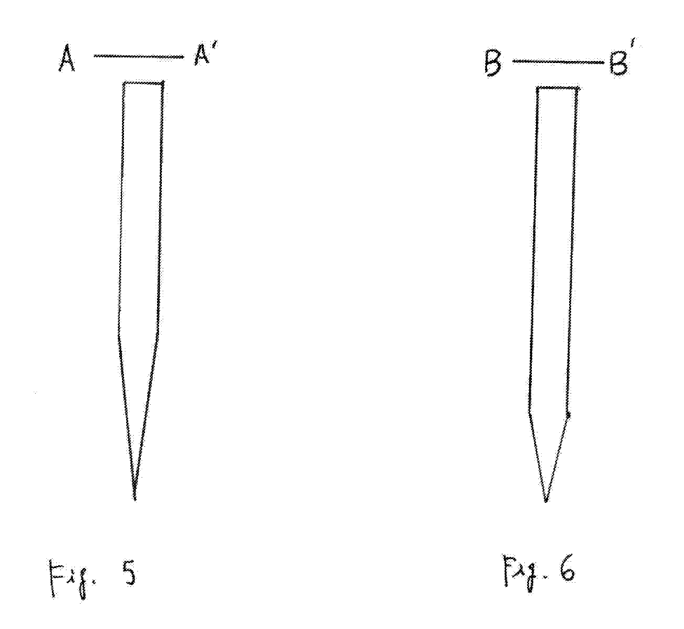

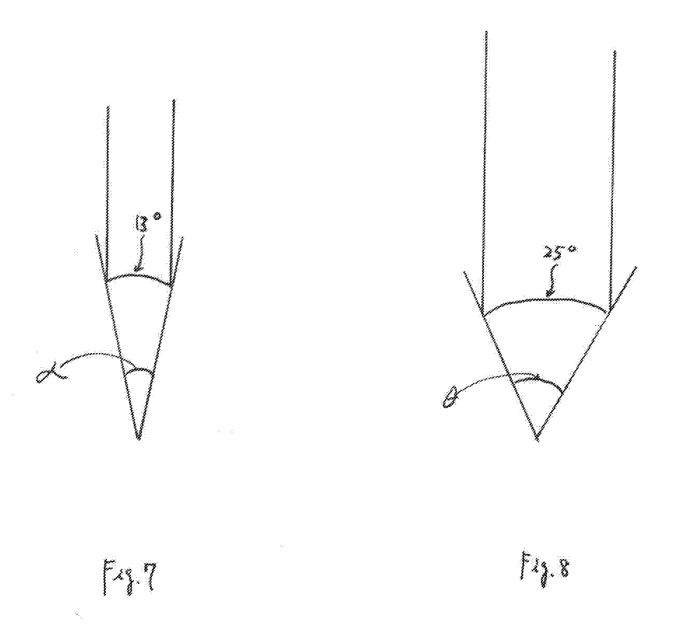

The edge (3) forms an acute cutting angle α and θ depends on the location. FIG. 5 is a cross-sectional view of the skin surgery knife of FIG. 4 along section line A-A′ and FIG. 6 is a cross-sectional view of the skin surgery knife of FIG. 4 along section line B-B′. The cutting edge (4) has the angle (α) and the cutting edge (5) has angle (θ). The angle (α) is smaller than the angle (θ). As a result the skin surgery knife has a blunt cutting edge angle of section near the point of the blade was provided. The cutting edge angle (α) of section near the handle (2) is more sharper and the surface is more even. The difference between the angle (α) and (θ) is at least ten degrees. FIG. 7 is an enlarged, cross-sectional view of cutting edge portion of the skin surgery knife of FIG. 5 and FIG. 8 is an enlarged, cross-sectional view of cutting edge portion of the skin surgery knife of FIG. 6.

In the section near the handle (2), the cutting angle (α) is, in an exemplary embodiment, between seven degrees (7°) and fifteen degrees (15°), in particular, thirteen degrees (13°). In the section near the point (7) of the blade, the cutting angle (θ) is, between fifteen degrees (15°) and thirty-five degrees (35°), preferably twenty-five degree (25°). The continuous change in the cutting angle is effected such that the width of the facet 6 remains substantially uniform along the length of the edge 3. This feature is also achieved due to the fact that, at least in the area near the edge (3), the blade (1) decreases in thickness from the section near the blade point (7) to the handle (2).

As shown before, the skin surgery knife of the current invention enables a quicker closing of incision mark.

1. A skin surgery knife, scalpel, comprising: a handle; and a blade having a point, sides, and an edge ground on both of the sides to form a cutting edge, the cutting edge: having a section near the handle and a section near the point of the blade; and having an acute cutting angle that becomes continuously bigger from the section near the handle towards the section near the point of the blade.

2. The skin surgery knife according to claim 1, wherein the cutting angle defines a largest cutting angle and a smallest cutting angle.

3. The skin surgery knife according to claim 1, wherein the cutting angle is between 10 and 20 degrees in the section near the handle; and is between 15 and 35 degrees in the section near the point of the blade.
